SEQUENCE LISTING (4 sequences)

SEQ ID NO: 1 - DNA, 50 bp, Artificial Sequence
  Description of Artificial Sequence: Synthetic oligonucleotide
caatggccaaagcactgatagtccgaaccttctgctgctcatccacaaga

SEQ ID NO: 2 - DNA, 50 bp, Artificial Sequence
  Description of Artificial Sequence: Synthetic oligonucleotide
caatggccaaagcactgatagtccgaacttcctgctgctcatccacaaga

SEQ ID NO: 3 - DNA, 20 bp, Artificial Sequence
  Description of Artificial Sequence: Synthetic oligonucleotide
agcaatggccaaagcactga

SEQ ID NO: 4 - DNA, 9292 bp, Artificial Sequence
  Description of Artificial Sequence: Synthetic polynucleotide
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgagcaatggccaaagcactgagttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaaggaattcggcagtggagagggcagaggaagtctgctaacatgcggtgacgtcgaggagaatcctggcccagtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaaggaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

(19) United States
(12) Patent Application Publication
(10) Pub. No.: US 2023/0218659 A1
(43) Pub. Date: Jul. 13, 2023

(54) Selenium-Based Compositions and Therapeutic Methods

(71) Applicant: Hess Pharmaceuticals, Inc, New York City, NY (US)

(72) Inventor: Jacob S.J. Hess, New York City, NY (US)

(73) Assignee: Hess Pharmaceuticals, Inc., New York City, NY (US)

(21) Appl. No.: 17/999,233

(22) PCT Filed: May 19, 2021
(86) PCT No.: PCT/US2021/033131

§ 371 Date: Nov. 18, 2022

Related U.S. Application Data
(60) Provisional application No. 63/027,399, filed on May 20, 2020.

(51) Int. Cl. A61K 33/04; A61P 7/00; A61P 35/00

(52) U.S. Cl. CPC A61K 33/04; A61P 7/00; A61P 35/00

(57) ABSTRACT

This invention provides selenium-based methods for treating refractory anemia with ring sideroblasts (RARS), cancer characterized by one or more genetic mutations associated with ring sideroblasts, and porphyria. This invention also provides related prophylactic methods, and articles of manufacture.

This application claims the benefit of U.S. Provisional Application No. 63/027,399, filed May 20, 2020, the contents of which are incorporated herein by reference.

Throughout this application, various publications are cited. The disclosure of these publications is hereby incorporated by reference into this application to describe more fully the state of the art to which this invention pertains.

### BACKGROUND OF THE INVENTION

### Selenium Generally

Selenium is a trace element and an essential nutrient for humans. It is naturally present in many foods, added to others, and available as a dietary supplement. It is also in more than two-dozen selenoproteins that play critical roles in reproduction, thyroid hormone metabolism, DNA synthesis, and protection from oxidative damage and infection (Sunde, et al. (2012)).

There are two forms of selenium - inorganic (selenate and selenite) and organic (selenomethionine and selenocysteine) (Sunde, et al. (2006)). Both forms are good dietary sources of selenium (Terry, et al.).

Plasma and serum selenium concentrations are the most commonly used measures of selenium status (Sunde, et al. (2012)). Plasma or serum selenium concentrations of eight micrograms (µg)/dL or higher in healthy people typically meet needs for selenoprotein synthesis (Sunde (2010)).

### Selenium and Cancer

Selenium might play a role in the prevention of cancer (Sunde, et al. (2006); Rayman; Allen, et al.; Combs, et al.). Studies have suggested an inverse association between selenium status and the risk of colorectal, prostate, lung, bladder, skin, esophageal, and gastric cancers (Dennert, et al.). A meta-analysis of 20 epidemiologic studies showed a potential inverse association between toenail, serum, and plasma selenium levels and prostate cancer risk (Brinkman, et al.).

Randomized controlled trials of selenium supplementation for cancer prevention have yielded conflicting results. The authors of a Cochrane review concluded, based on nine randomized clinical trials, that selenium might help prevent gastrointestinal cancers but noted that these results need to be confirmed in more appropriately designed randomized clinical trials (Bjelakovic, et al.). A secondary analysis of the double-blind, randomized, controlled Nutritional Prevention of Cancer Trial in 1,312 U.S. adults with a history of basal cell or squamous cell carcinomas of the skin found that 200 µg/day selenium as high-selenium baker’s yeast for six years was associated with a 52% to 65% lower risk of prostate cancer (Duffield-Lillico, et al.). This effect was strongest in men in the lowest tertile of selenium concentrations who had a baseline prostate-specific antigen (PSA) level of 4 ng/mL or lower. The Selenium and Vitamin E Cancer Prevention Trial (SELECT), a randomized, controlled trial in 35,533 men aged 50 years or older from the United States, Canada, and Puerto Rico, was discontinued after 5.5 years when analyses showed no association between prostate cancer risk and supplementation with 200 µg/day selenium with or without 400 international units (IU)/day vitamin E (Lippman, et al.). An additional 1.5 years of follow-up data on participants after they stopped taking the study supplements confirmed the lack of a significant association between selenium supplementation and prostate cancer risk (Klein, et al.).

In 2003, the FDA allowed a qualified health claim on foods and dietary supplements containing selenium to state that while “some scientific evidence suggests that consumption of selenium may reduce the risk of certain forms of cancer... FDA has determined that this evidence is limited and not conclusive” (Letters of Enforcement Discretion). More research is needed to confirm a relationship between selenium concentrations and cancer risk and to determine whether selenium supplements can help prevent any form of cancer.

### Risks From Excessive Selenium

Chronically high intakes of the organic and inorganic forms of selenium have similar effects (IMFN Board). Early indicators of excess intake are a garlic odor in the breath and a metallic taste in the mouth. The most common clinical signs of chronically high selenium intakes, or selenosis, are hair and nail loss or brittleness. Other symptoms include lesions of the skin and nervous system, nausea, diarrhea, skin rashes, mottled teeth, fatigue, irritability, and nervous system abnormalities.

Acute selenium toxicity can cause severe gastrointestinal and neurological symptoms, acute respiratory distress syndrome, myocardial infarction, hair loss, muscle tenderness, tremors, lightheadedness, facial flushing, kidney failure, cardiac failure, and, in rare cases, death (Sunde, et al. (2006); IMFN Board).

### SUMMARY OF THE INVENTION

This invention provides a method for treating a subject having refractory anemia with ring sideroblasts (RARS) comprising administering to the subject an effective amount of a selenium-based agent.

This invention also provides a method for treating a subject having a cancer characterized by one or more genetic mutations associated with ring sideroblasts, comprising administering to the subject an effective amount of a selenium-based agent.

This invention further provides a method for treating a subject having porphyria comprising administering to the subject an effective amount of a selenium-based agent. This invention still further provides a method for inhibiting the exacerbation of porphyria in a subject to whom a therapeutic agent is administered, wherein the subject’s cells possess one or more mutations in the heme biosynthesis pathway that render the subject susceptible to porphyria exacerbation by the therapeutic agent, which method comprises administering to the subject an effective amount of a selenium-based agent before, during, and/or after administering the therapeutic agent.

This invention also provides a method for treating a human subject having RARS comprising orally administering to the subject an effective amount of sodium selenite.

This invention further provides an article of manufacture comprising (i) a selenium-based agent, (ii) a pharmaceutically acceptable carrier, and (iii) a label indicating a use for an admixture of the agent and carrier in treating a subject afflicted with RARS.

Finally, this invention provides an article of manufacture comprising (i) sodium selenite, (ii) a pharmaceutically acceptable carrier, and (iii) a label indicating a use for an admixture of the sodium selenite and carrier in treating a human subject having RARS.

### BRIEF DESCRIPTION OF THE FIGURES

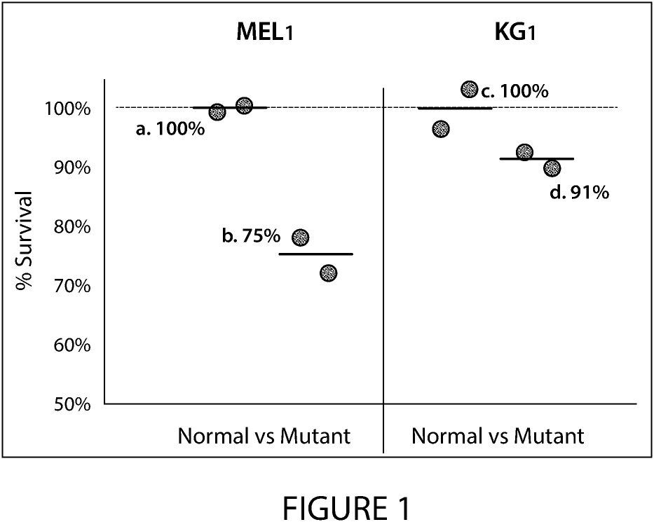

### FIG. 1

This figure shows the survival of melanoma and leukemia cells transfected with the K700E splicing mutation. When melanoma and leukemia cells, genetically manipulated by transfection with the splicing factor mutation K700E, are grown in the presence of sodium selenite, sodium selenite has a cytotoxic effect against the mutated cells.

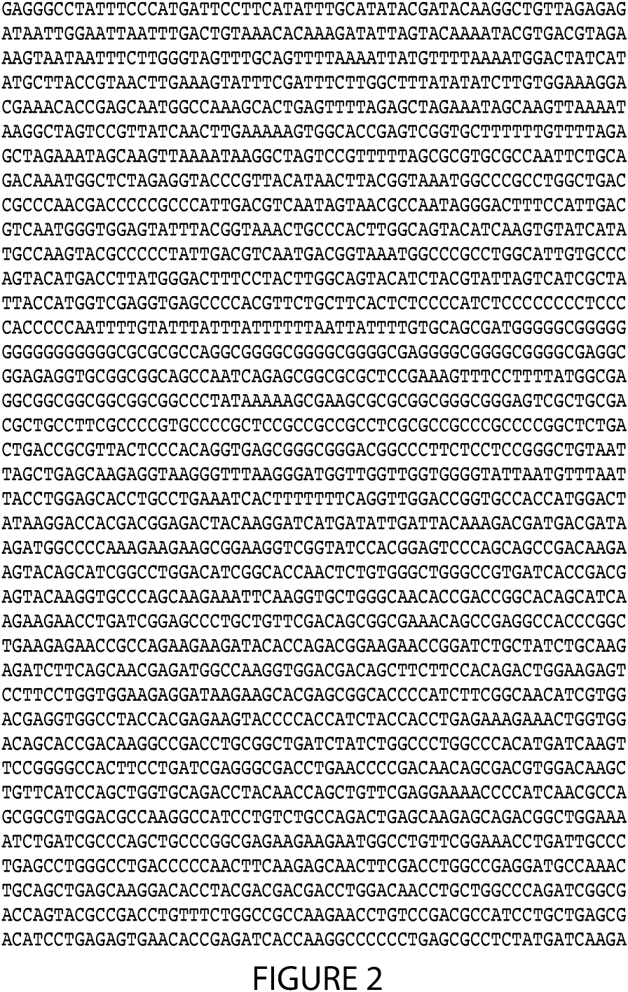

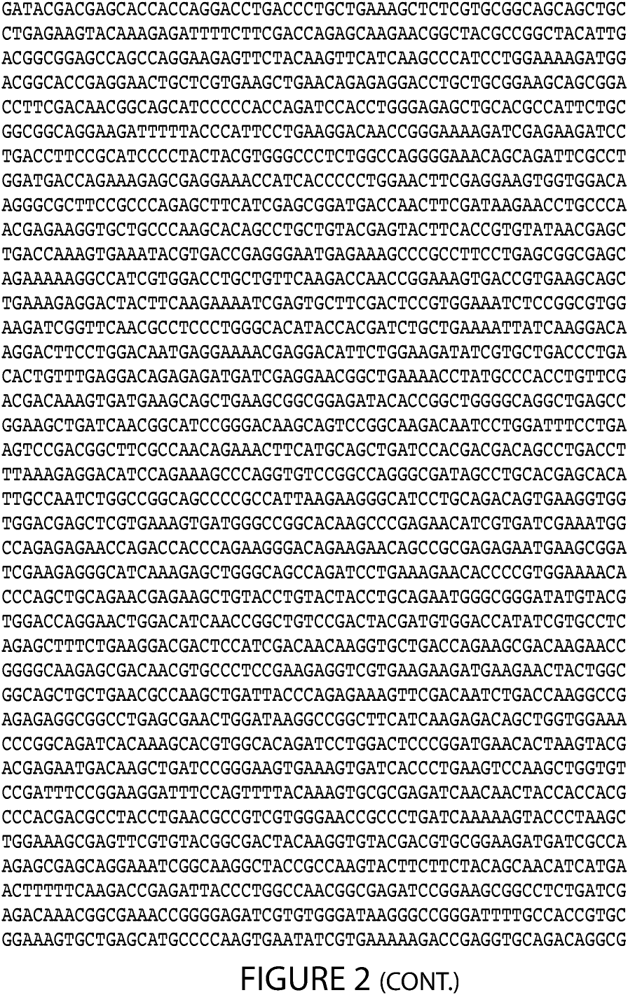

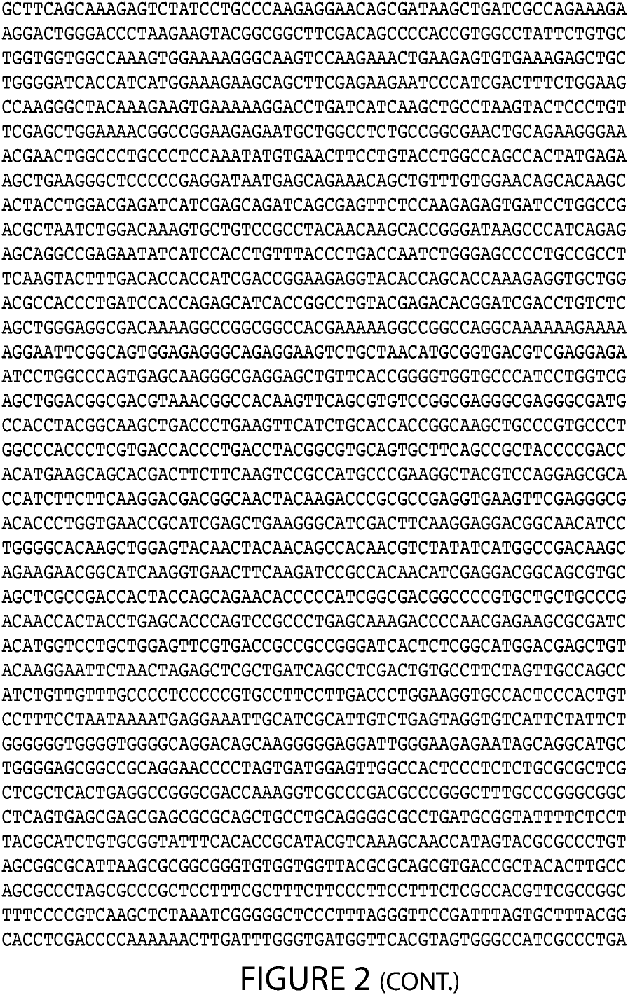

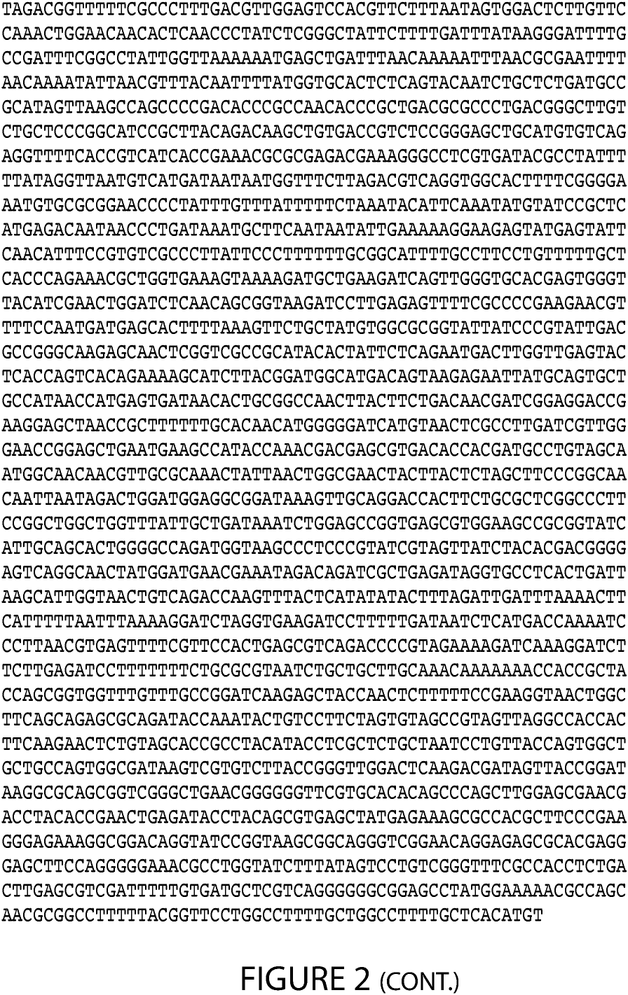

### FIG. 2

This figure shows the Cas9 plasmid sequence with gRNA, px458 PAM1_pSpCas9 BB-2A-GFP (PX458).

### DETAILED DESCRIPTION OF THE INVENTION

### Definitions

In this application, certain terms are used which shall have the meanings set forth as follows.

As used herein, “administer”, with respect to an agent, means to deliver the agent to a subject’s body via any known method. Specific modes of administration include, without limitation, intravenous, intramuscular, oral, sublingual, transdermal, subcutaneous, intratumoral, intraperitoneal, and intrathecal administration. Preferred in this invention is oral and intravenous administration.

In addition, in this invention, the various agents can be formulated using one or more routinely used pharmaceutically acceptable carriers. Such carriers are well known to those skilled in the art. For example, oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Injectable drug delivery systems include, for example, solutions, suspensions, gels, microspheres and polymeric injectables, and can comprise excipients such as solubility-altering agents (e.g., ethanol, propylene glycol and sucrose) and polymers (e.g., polycaprylactones and PLGA’s). Implantable systems include rods and discs and can contain excipients such as PLGA and polycaprylactone.

As used herein, an amount of a selenium-based agent (e.g., sodium selenite pentahydrate) “effective” to treat a subject having RARS includes, without limitation, (i) 100 µg, 200 µg, 300 µg, 400 µg, 500 µg, 600 µg, 700 µg, 800 µg, 900 µg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, and 150 mg; (ii) 100-200 µg, 200-300 µg, 300-400 µg, 400-500 µg, 500-600 µg, 600-700 µg, 700-800 µg, 800-900 µg, 900-1,000 µg, 1.0-1.5 mg, 1.5-2.0 mg, 2.0-2.5 mg, 2.5-3.0 mg, 3.0-3.5 mg, 3.5-4.0 mg, 4.0-4.5 mg, 4.5-5.0 mg, 5.0-6.0 mg, 6.0-7.0 mg, 7.0-8.0 mg, 8.0-9.0 mg, 9.0-10 mg, 10-15 mg, 15-20 mg, 20-25 mg, 25-30 mg, 30-35 mg, 35-40 mg, 40-45 mg, 45-50 mg, 50-60 mg, 60-70 mg, 70-80 mg, 80-90 mg, 90-100 mg, 100-110 mg, 110-120 mg, 120-130 mg, 130-140 mg, and 140-150 mg; (iii) 1 µg/kg, 2 µg/kg, 3 pg/kg, 4 µg/kg, 5 µg/kg, 6 µg/kg, 7 µg/kg, 8 µg/kg, 9 µg/kg, 10 µg/kg, 15 µg/kg, 20 µg/kg, 25 µg/kg, 30 µg/kg, 35 µg/kg, 40 µg/kg, 50 ug/kg, 60 µg/kg, 70 µg/kg, 80 µg/kg, 90 µg/kg, 100 µg/kg, 150 µg/kg, 200 µg/kg, 250 µg/kg, 300 µg/kg, 350 µg/kg, 400 µg/kg, 450 µg/kg, 500 µg/kg, 600 µg/kg, 700 µg/kg, 800 µg/kg, 900 µg/kg, 1,000 µg/kg, 1,100 µg/kg, 1,200 µg/kg, 1,300 µg/kg, 1,400 µg/kg, and 1,500 µg/kg; and (iv) 1-2 µg/kg, 2-3 µg/kg, 3-4 µg/kg, 4-5 µg/kg, 5-6 µg/kg, 6-7 µg/kg, 7-8 µg/kg, 8-9 µg/kg, 9-10 µg/kg, 10-15 µg/kg, 15-20 µg/kg, 20-25 µg/kg, 25-30 µg/kg, 30-35 µg/kg, 35-40 µg/kg, 40-50 µg/kg, 50-60 µg/kg, 60-70 µg/kg, 70-80 µg/kg, 80-90 µg/kg, 90-100 µg/kg, 100-150 µg/kg, 150-200 µg/kg, 200-250 µg/kg, 250-300 µg/kg, 300-350 µg/kg, 350-400 µg/kg, 400-450 µg/kg, 450-500 µg/kg, 500-600 µg/kg, 600-700 µg/kg, 700-800 µg/kg, 800-900 µg/kg, 900-1,000 µg/kg, 1,000-1,100 µg/kg, 1,100-1,200 µg/kg, 1,200-1,300 µg/kg, 1,300-1,400 µg/kg, and 1,400-1,500 µg/kg. In one embodiment, the effective amount of agent is administered as a single, one-time-only dose. In another embodiment, the effective amount of agent is administered as two or more doses over a period of days, weeks, or months (e.g., twice daily for one or two weeks; once daily for one or two weeks; every other day for two weeks; three times per week for two weeks; twice per week for two weeks; once per week for two weeks; or twice with the administrations separated by two weeks).

As used herein, an amount of a selenium-based agent (e.g., sodium selenite pentahydrate) “effective” to treat a subject having cancer includes, without limitation, (i) 100 µg, 200 µg, 300 µg, 400 µg, 500 µg, 600 µg, 700 µg, 800 µg, 900 µg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, and 150 mg; (ii) 100-200 µg, 200-300 µg, 300-400 µg, 400-500 µg, 500-600 µg, 600-700 µg, 700-800 µg, 800-900 µg, 900-1,000 µg, 1.0-1.5 mg, 1.5-2.0 mg, 2.0-2.5 mg, 2.5-3.0 mg, 3.0-3.5 mg, 3.5-4.0 mg, 4.0-4.5 mg, 4.5-5.0 mg, 5.0-6.0 mg, 6.0-7.0 mg, 7.0-8.0 mg, 8.0-9.0 mg, 9.0-10 mg, 10-15 mg, 15-20 mg, 20-25 mg, 25-30 mg, 30-35 mg, 35-40 mg, 40-45 mg, 45-50 mg, 50-60 mg, 60-70 mg, 70-80 mg, 80-90 mg, 90-100 mg, 100-110 mg, 110-120 mg, 120-130 mg, 130-140 mg, and 140-150 mg; (iii) 1 µg/kg, 2 µg/kg, 3 µg/kg, 4 µg/kg, 5 µg/kg, 6 µg/kg, 7 µg/kg, 8 µg/kg, 9 µg/kg, 10 µg/kg, 15 µg/kg, 20 µg/kg, 25 µg/kg, 30 µg/kg, 35 µg/kg, 40 µg/kg, 50 µg/kg, 60 µg/kg, 70 µg/kg, 80 µg/kg, 90 µg/kg, 100 µg/kg, 150 µg/kg, 200 µg/kg, 250 µg/kg, 300 µg/kg, 350 µg/kg, 400 µg/kg, 450 µg/kg, 500 µg/kg, 600 µg/kg, 700 µg/kg, 800 µg/kg, 900 µg/kg, 1,000 µg/kg, 1,100 µg/kg, 1,200 µg/kg, 1,300 µg/kg, 1,400 µg/kg, and 1,500 µg/kg; and (iv) 1-2 µg/kg, 2-3 µg/kg, 3-4 µg/kg, 4-5 µg/kg, 5-6 µg/kg, 6-7 µg/kg, 7-8 µg/kg, 8-9 µg/kg, 9-10 µg/kg, 10-15 µg/kg, 15-20 µg/kg, 20-25 µg/kg, 25-30 µg/kg, 30-35 µg/kg, 35-40 µg/kg, 40-50 µg/kg, 50-60 µg/kg, 60-70 µg/kg, 70-80 µg/kg, 80-90 µg/kg, 90-100 µg/kg, 100-150 µg/kg, 150-200 µg/kg, 200-250 µg/kg, 250-300 µg/kg, 300-350 µg/kg, 350-400 µg/kg, 400-450 µg/kg, 450-500 µg/kg, 500-600 µg/kg, 600-700 µg/kg, 700-800 µg/kg, 800-900 µg/kg, 900-1,000 µg/kg, 1,000-1,100 µg/kg, 1,100-1,200 µg/kg, 1,200-1,300 µg/kg, 1,300-1,400 µg/kg, and 1,400-1,500 µg/kg. In one embodiment, the effective amount of agent is administered as a single, one-time-only dose. In another embodiment, the effective amount of agent is administered as two or more doses over a period of days, weeks, or months (e.g., twice daily for one or two weeks; once daily for one or two weeks; every other day for two weeks; three times per week for two weeks; twice per week for two weeks; once per week for two weeks; or twice with the administrations separated by two weeks).

As used herein, an amount of a selenium-based agent (e.g., sodium selenite pentahydrate) “effective” to treat a subject having porphyria includes, without limitation, (i) 100 µg, 200 µg, 300 µg, 400 µg, 500 µg, 600 µg, 700 µg, 800 µg, 900 µg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, and 150 mg; (ii) 100-200 µg, 200-300 µg, 300-400 µg, 400-500 µg, 500-600 µg, 600-700 µg, 700-800 µg, 800-900 µg, 900-1,000 µg, 1.0-1.5 mg, 1.5-2.0 mg, 2.0-2.5 mg, 2.5-3.0 mg, 3.0-3.5 mg, 3.5-4.0 mg, 4.0-4.5 mg, 4.5-5.0 mg, 5.0-6.0 mg, 6.0-7.0 mg, 7.0-8.0 mg, 8.0-9.0 mg, 9.0-10 mg, 10-15 mg, 15-20 mg, 20-25 mg, 25-30 mg, 30-35 mg, 35-40 mg, 40-45 mg, 45-50 mg, 50-60 mg, 60-70 mg, 70-80 mg, 80-90 mg, 90-100 mg, 100-110 mg, 110-120 mg, 120-130 mg, 130-140 mg, and 140-150 mg; (iii) 1 ug/kg, 2 ug/kg, 3 µg/kg, 4 ug/kg, 5 µg/kg, 6 µg/kg, 7 µg/kg, 8 µg/kg, 9 µg/kg, 10 µg/kg, 15 µg/kg, 20 µg/kg, 25 µg/kg, 30 µg/kg, 35 µg/kg, 40 µg/kg, 50 µg/kg, 60 µg/kg, 70 µg/kg, 80 µg/kg, 90 µg/kg, 100 µg/kg, 150 µg/kg, 200 µg/kg, 250 µg/kg, 300 µg/kg, 350 µg/kg, 400 µg/kg, 450 µg/kg, 500 µg/kg, 600 µg/kg, 700 µg/kg, 800 µg/kg, 900 µg/kg, 1,000 µg/kg, 1,100 µg/kg, 1,200 µg/kg, 1,300 µg/kg, 1,400 µg/kg, and 1,500 µg/kg; and (iv) 1-2 µg/kg, 2-3 µg/kg, 3-4 µg/kg, 4-5 µg/kg, 5-6 µg/kg, 6-7 µg/kg, 7-8 µg/kg, 8-9 µg/kg, 9-10 µg/kg, 10-15 µg/kg, 15-20 µg/kg, 20-25 µg/kg, 25-30 µg/kg, 30-35 µg/kg, 35-40 µg/kg, 40-50 µg/kg, 50-60 ug/kg, 60-70 µg/kg, 70-80 µg/kg, 80-90 µg/kg, 90-100 µg/kg, 100-150 µg/kg, 150-200 µg/kg, 200-250 µg/kg, 250-300 µg/kg, 300-350 µg/kg, 350-400 µg/kg, 400-450 µg/kg, 450-500 µg/kg, 500-600 µg/kg, 600-700 µg/kg, 700-800 µg/kg, 800-900 µg/kg, 900-1,000 µg/kg, 1,000-1,100 µg/kg, 1,100-1,200 µg/kg, 1,200-1,300 µg/kg, 1,300-1,400 µg/kg, and 1,400-1,500 µg/kg. In one embodiment, the effective amount of agent is administered as a single, one-time-only dose. In another embodiment, the effective amount of agent is administered as two or more doses over a period of days, weeks, or months (e.g., twice daily for one or two weeks; once daily for one or two weeks; every other day for two weeks; three times per week for two weeks; twice per week for two weeks; once per week for two weeks; or twice with the administrations separated by two weeks).

As used herein, an amount of a selenium-based agent (e.g., sodium selenite pentahydrate) “effective” to inhibit the exacerbation of porphyria includes, without limitation, (i) 100 µg, 200 µg, 300 µg, 400 µg, 500 µg, 600 µg, 700 µg, 800 µg, 900 µg, 1.0 mg, 1.5 mg, 2.0 mg, 2.5 mg, 3.0 mg, 3.5 mg, 4.0 mg, 4.5 mg, 5.0 mg, 6.0 mg, 7.0 mg, 8.0 mg, 9.0 mg, 10 mg, 15 mg, 20 mg, 25 mg, 30 mg, 35 mg, 40 mg, 45 mg, 50 mg, 60 mg, 70 mg, 80 mg, 90 mg, 100 mg, 110 mg, 120 mg, 130 mg, 140 mg, and 150 mg; (ii) 100-200 µg, 200-300 µg, 300-400 µg, 400-500 µg, 500-600 µg, 600-700 µg, 700-800 µg, 800-900 µg, 900-1,000 µg, 1.0-1.5 mg, 1.5-2.0 mg, 2.0-2.5 mg, 2.5-3.0 mg, 3.0-3.5 mg, 3.5-4.0 mg, 4.0-4.5 mg, 4.5-5.0 mg, 5.0-6.0 mg, 6.0-7.0 mg, 7.0-8.0 mg, 8.0-9.0 mg, 9.0-10 mg, 10-15 mg, 15-20 mg, 20-25 mg, 25-30 mg, 30-35 mg, 35-40 mg, 40-45 mg, 45-50 mg, 50-60 mg, 60-70 mg, 70-80 mg, 80-90 mg, 90-100 mg, 100-110 mg, 110-120 mg, 120-130 mg, 130-140 mg, and 140-150 mg; (iii) 1 ug/kg, 2 ug/kg, 3 ug/kg, 4 µg/kg, 5 µg/kg, 6 µg/kg, 7 µg/kg, 8 µg/kg, 9 µg/kg, 10 µg/kg, 15 µg/kg, 20 µg/kg, 25 µg/kg, 30 µg/kg, 35 µg/kg, 40 µg/kg, 50 ug/kg, 60 µg/kg, 70 µg/kg, 80 µg/kg, 90 µg/kg, 100 µg/kg, 150 µg/kg, 200 µg/kg, 250 µg/kg, 300 µg/kg, 350 µg/kg, 400 µg/kg, 450 µg/kg, 500 µg/kg, 600 µg/kg, 700 µg/kg, 800 µg/kg, 900 µg/kg, 1,000 µg/kg, 1,100 µg/kg, 1,200 µg/kg, 1,300 µg/kg, 1,400 µg/kg, and 1,500 µg/kg; and (iv) 1-2 µg/kg, 2-3 µg/kg, 3-4 µg/kg, 4-5 µg/kg, 5-6 µg/kg, 6-7 µg/kg, 7-8 µg/kg, 8-9 µg/kg, 9-10 µg/kg, 10-15 µg/kg, 15-20 µg/kg, 20-25 µg/kg, 25-30 µg/kg, 30-35 µg/kg, 35-40 µg/kg, 40-50 µg/kg, 50-60 µg/kg, 60-70 µg/kg, 70-80 µg/kg, 80-90 µg/kg, 90-100 µg/kg, 100-150 µg/kg, 150-200 µg/kg, 200-250 µg/kg, 250-300 µg/kg, 300-350 µg/kg, 350-400 µg/kg, 400-450 µg/kg, 450-500 µg/kg, 500-600 µg/kg, 600-700 µg/kg, 700-800 µg/kg, 800-900 µg/kg, 900-1,000 µg/kg, 1,000-1,100 µg/kg, 1,100-1,200 µg/kg, 1,200-1,300 µg/kg, 1,300-1,400 µg/kg, and 1,400-1,500 µg/kg. In one embodiment, the effective amount of agent is administered as a single, one-time-only dose. In another embodiment, the effective amount of agent is administered as two or more doses over a period of days, weeks, or months (e.g., twice daily for one or two weeks; once daily for one or two weeks; every other day for two weeks; three times per week for two weeks; twice per week for two weeks; once per week for two weeks; or twice with the administrations separated by two weeks).

As used herein, a therapeutic agent that can “exacerbate porphyria” in a subject whose cells possess one or more mutations in the heme biosynthesis pathway includes, without limitation, the following therapeutic agents: (i) sulfonamides (e.g., sulfadiazine, sulfasalazine, and trimethoprim/sulfamethoxazole); (ii) sulfonylureas (e.g., glibenclamide, gliclazide, and glimepiride); (iii) barbiturates (e.g., thiopental, phenobarbital, and primidone); (iv) antifungals (e.g., fluconazole, griseofulvin, ketoconazole, and voriconazole); (v) antibiotics (e.g., rifapentine, rifampicin, rifabutine, isoniazid, nitrofurantoin, and metronidazole); (vi) ergot derivatives (e.g., dihydroergotamine, ergometrine, ergotamine, and methysergide); (vii) antiretroviral medications (e.g., indinavir, nevirapine, ritonavir, and saquinavir); (viii) progestogens; (ix) anticonvulsants (e.g., carbamazepine, ethosuximide, phenytoin, topiramate, and valproate); (x) painkillers (e.g., dextropropoxyphene, ketorolac, metamizole, and pentazocine); (xi) anti-cancer drugs (e.g., bexarotene, busulfan, chlorambucil, estramustine, etoposide, flutamide, idarubicin, ifosfamide, irinotecan, ixabepilone, letrozole, lomustine, megestrol, mitomycin, mitoxantrone, paclitaxel, procarbazine, tamoxifen, and topotecan); (xii) antidepressants (e.g., imipramine, phenelzine, and trazodone); (xiii) antipsychotics (e.g., risperidone and ziprasidone); (xiv) retinoids (e.g., acitretin and isotretinoin); (xv) cocaine; (xvi) methyldopa; (xvii) fenfluramine; (xviii) disulfiram; (xix) orphenadrine; (xx) pentoxifylline; and (xxi) sodium aurothiomalate.

As used herein, “inhibiting the exacerbation” of porphyria in a subject includes, without limitation, reducing the likelihood of the disorder’s exacerbation by at least 10%, at least 20%, at least 30%, at least 40%, at least 50%, at least 60%, at least 70%, at least 80%, at least 90%, or at least 95%. Preferably, inhibiting the disorder’s exacerbation in a subject means preventing such exacerbation.

As used herein, the term “PARS” means refractory anemia with ring sideroblasts. RARS is a type of MDS and is also known as MDS-RS, where MDS stands for myelodysplastic syndrome. Causes of ringed sideroblasts include, without limitation, clonal diseases and non-clonal conditions. Clonal diseases include (1) myelodysplastic syndromes (MDS) (including (i) RARS, (ii) refractory cytopenias with multilineage dysplasia and ringed sideroblasts (RCMD-RS), (iii) refractory anemia with excess blasts-½ (RAEB-½), and (iv) unclassifiable MDS (MDS-U)); (2) myeloproliferative neoplasms (MPN) (including (i) essential thrombocythemia (ET), (ii) primary myelofibrosis (PMF), and (iii) polycythemia vera); and (3) MDS/MPN overlap syndromes (including (i) MDS/MPN with ringed sideroblasts and thrombocytosis (MDS/MPN-RS-T), (ii) chronic myelomonocytic leukemia (CMML), and (iii) unclassified MDS/MPN). Non-clonal conditions include (1) hereditary sideroblastic anemia (including (i) defects in heme synthesis (e.g., XLSA (X-linked sideroblastic anemia, with mutations in ALAS2 and SLC25A38)), (ii) defects in Fe—S cluster biogenesis and protein synthesis (e.g., XLSA/A (X-linked sideroblastic anemia with ataxia, with mutations in ABCB7 and GLRX5)), (iii) defects in mitochondrial respiration (e.g., MLASA (mitochondrial myopathy, lactic acidosis, and SA, with mutations in PUS1, YARS2, MT-ATP6, LARS2), PMPS (Pearson’s marrow-pancreas syndrome, with deletions, rearrangements, or duplications of mitochondrial DNA), SIFD (congenital SA associated with B-cell immunodeficiency, periodic fevers, and developmental delay, with mutations in TRNT1), NDUFB11-mutated sideroblastic anemia, HSPA9-mutated sideroblastic anemia, and FECH-mutated associated erythropoietic protoporphyria (EPP)), and (iv) thiamine responsive megaloblastic anemia syndrome, mutated SLC19A2); and (2) acquired conditions (including (i) excess alcohol, (ii) drug exposure (isoniazid, chloramphenicol, linezolid, and penicillamine), (iii) lead, zinc toxicity, and (iv) copper deficiency).

As used herein, the term “selenium-based agent” includes, without limitation, selenate, a selenate salt (e.g., sodium selenate, cupric selenate, magnesium selenate, barium selenate, potassium selenate, and cobalt selenate), selenite, a selenite salt (e.g., sodium selenite (including anhydrous sodium selenite and, preferably, sodium selenite pentahydrate), cupric selenite, cobalt selenite, potassium selenite), selenocysteine, selenomethionine, phenylenebis(methylene) selenocyanate, selenium oxide, and a mannose-6 derivative (e.g., mannose-6-phosphate-selenate and mannose-6-phosphate-selenite (see, e.g., U.S. Pat. No. 8,399,657)). Additional selenium-based agents include, without limitation, a selenium-bound antibody (e.g., a selenium-bound anti-CD33 antibody such as mylotarg), a selenium-bound peptide (e.g., selenium bound to luteinizing hormone-releasing hormone, to D-Lys6-LHRH, or to angiopep-2), a selenium-bound organic acid (e.g., selenium-bound folic acid, selenium-bound citric acid, and selenium-bound ascorbic acid), and a selenium-bound carbohydrate (e.g., selenium bound to chitosan or hyaluronic acid). These carrier molecules (e.g., antibodies, peptides, and carbohydrates) can also be used, for example, in conjunction with nanosphere constructions, where the spheres contain the selenium-based agent and are coated with these carrier molecules for targeted delivery of high payloads.

As used herein, the term “subject” includes, without limitation, a mammal such as a human, a non-human primate, a dog, a cat, a horse, a sheep, a goat, a cow, a rabbit, a pig, a hamster, a rat, and a mouse. A human subject can be of any age, gender, or state of co-morbidity. In one embodiment, the subject is male, and in another, the subject is female. In one embodiment, the subject is younger than 60 years old. In another embodiment, the subject is between 0-6 months old, 7-12 months old, 1-3 years old, 4-8 years old, 9-13 years old, 14-18 years old, and 19-59 years old. In a further embodiment, the subject is at least 60 years old, at least 65 years old, at least 70 years old, at least 75 years old, at least 80 years old, at least 85 years old, or at least 90 years old.

As used herein, “treating” a subject afflicted with a disorder (e.g., a subject having RARS and symptomatic of that disorder) includes, without limitation, (i) slowing, stopping, or reversing the progression of one or more of the disorder’s symptoms, (ii) slowing, stopping or reversing the progression of the disorder’s underlying such symptoms, (iii) reducing or eliminating the likelihood of the symptoms’ recurrence, and/or (iv) slowing the progression of, lowering, or eliminating the disorder. In the preferred embodiment, treating a subject afflicted with a disorder includes (i) reversing the progression of one or more of the disorder’s symptoms, (ii) reversing the progression of the disorder underlying such symptoms, (iii) preventing the symptoms’ recurrence, and/or (iv) eliminating the disorder. In a further embodiment, treating a subject afflicted with a disorder (e.g., a subject having RARS and symptomatic of that disorder) includes first exacerbating the disorder and/or its symptoms prior to ameliorating the disorder and/or its symptoms.

### Embodiments of the Invention

This invention provides certain therapeutic and prophylactic methods employing selenium-based agents. First, this invention provides a method for treating a subject having refractory anemia with ring sideroblasts (RARS), comprising administering to the subject an effective amount of a selenium-based agent (e.g., sodium selenite pentahydrate). In a preferred embodiment of this method, the selenium-based agent is sodium selenite, and the subject is human. Clinical endpoints for treating RARS include, without limitation, one or more of the following: (i) a substantial decrease or elimination of ring sideroblasts in the bone marrow as detected by Prussian stain (which is the standard clinical method of detection); (ii) a noticeable relief of fatigue caused by anemia; (iii) a decreased long-term prevalence of complications from iron overload (with the caveat that upon treatment initiation, there could be temporary increased fatigue and complications of iron overload); (iv) the loss of detectable mutated SF3B1; (v) the loss of ring sideroblasts as determined by immunohistochemical staining, and (vi) transfusion independence.

In a preferred embodiment, this method further comprises administering to the subject an effective amount of azacytidine. In that regard, this invention provides a composition comprising a selenium-based agent (e.g., sodium selenite pentahydrate) and azacytidine. In one embodiment, the azacytidine (either in conjunction with the selenium-based agent or as part of the same composition) is administered intravenously for seven days at 75 mg/m² every 28 days.

In another preferred embodiment, this method further comprises administering to the subject an effective amount of luspatercept. In that regard, this invention provides a composition comprising a selenium-based agent (e.g., sodium selenite pentahydrate) and luspatercept. In one embodiment, the luspatercept (either in conjunction with the selenium-based agent or as part of the same composition) is administered by subcutaneous injection at 1 mg/kg once every three weeks as needed.

In a further preferred embodiment, this method further comprises administering to the subject an effective amount of fludarabine phosphate, cyclophosphamide, and rituximab (collectively called FCR). In one embodiment, FCR is administered according to the following regimen. Cycle 1 of FCR consists of the following: rituximab 375 mg/m² IV at 50 mg/h (increase by 50 mg/h every 30 minutes until a target rate of 400 mg/h is reached) on day 1, plus cyclophosphamide 250 mg/m² IV over 10-30 minutes on days 1-3, plus fludarabine 25 mg/m² IV over 20-30 min on days 1-3. Cycles 2-6 of FCR differ from cycle 1 only in that the rituximab dose is increased from 375 mg/m² to 500 mg/m². Thus, the regimen is as follows: rituximab 500 mg/m² IV at 50 mg/h (increase by 50 mg/h every 30 minutes until a target rate of 400 mg/h is reached) on day 1, plus cyclophosphamide 250 mg/m² IV over 10-30 minutes on days 1-3, plus fludarabine 25 mg/m² IV over 20-30 minutes on days 1-3. In conjunction with FCR administration, the selenium-based agent (e.g., sodium selenite pentahydrate) is administered, for example, (i) intravenously as a saline solution immediately following FCR administration, (ii) orally on all days of FCR administration, or (iii) intravenously daily at high concentration for three days at the beginning of, or later during, FCR administration.

This invention also provides a method for treating a subject having a cancer characterized by one or more genetic mutations associated with ring sideroblasts, comprising administering to the subject an effective amount of a selenium-based agent. In a preferred embodiment of this method, the selenium-based agent is sodium selenite (e.g., sodium selenite pentahydrate), and the subject is human.

In another preferred embodiment of this method, the cancer is characterized by one or more mutations in the SF3B1 gene. Preferably, the cancer is characterized by one or more SF3B1 mutations selected from the group consisting of a K700 mutation (e.g., K700A, K700R, K700N, K700D, K700C, K700Q, K700E, K700G, K700H, K700I, K700L, K700M, K700F, K700P, K700S, K700T, K700W, K700Y, or K700V), a K666 mutation (e.g., K666A, K666R, K666N, K666D, K666C, K666Q, K666E, K666G, K666H, K666I, K666L, K666M, K666F, K666P, K666S, K666T, K666W, K666Y, or K666V), an H662 mutation (e.g., H662A, H662R, H662N, H662D, H662C, H662Q, H662E, H662G, H662H, H662I, H662L, H662M, H662F, H662P, H662S, H662T, H662W, H662Y, or H662V), an E622 mutation (e.g., E662A, E662R, E662N, E662D, E662C, E662Q, E662E, E662G, E662H, E662I, E662L, E662M, E662F, E662P, E662S, E662T, E662W, E662Y, or E662V), and an R625 mutation (e.g., R625A, R625R, R625N, R625D, R625C, R625Q, R625E, R625G, R625H, R625I, R625L, R625M, R625F, R625P, R625S, R625T, R625W, R625Y, or R625V). In a further embodiment of this method, the cancer is uveal melanoma, chronic lymphoid leukemia, melanoma, acute myeloid leukemia, breast cancer, pancreatic cancer, non-small cell lung cancer, or cervical cancer.

This invention further provides a method for treating a subject having porphyria comprising administering to the subject an effective amount of a selenium-based agent. In a preferred embodiment of this method, the selenium-based agent is sodium selenite (e.g., sodium selenite pentahydrate), and the subject is human.

In another preferred embodiment of this method, the porphyria is selected from the group consisting of porphyria cutanea tarda, acute intermittent porphyria, and erythropoietic protoporphyria.

This invention further provides a method for inhibiting the exacerbation of porphyria in a subject to whom a therapeutic agent is administered, wherein the subject’s cells possess one or more mutations in the heme biosynthesis pathway that render the subject susceptible to porphyria exacerbation by the therapeutic agent, which method comprises administering to the subject an effective amount of a selenium-based agent before, during, and/or after administering the therapeutic agent. In a preferred embodiment of this method, the selenium-based agent is sodium selenite (e.g., sodium selenite pentahydrate), and the subject is human.

Exacerbation of porphyria in a subject is characterized, for example, by one or more of the following: (i) detectability of the mutated gene; (ii) disease relapse; (iii) photosensitivity for those with X-linked dominant protoporphyria (XLDPP), congenital erythropoietic porphyria (CEP), porphyria cutanea tarda (PCT), harderoporphyria (HP), variegate porphyria (VP), and erythropoietic protoporphyria (EPP); and (iv) peripheral neuropathy for those with aminolevulinate dehydratase deficiency porphyria (ALADP) and acute intermittent porphyria (AIP). Accordingly, in one embodiment, clinical endpoints for inhibiting the exacerbation of porphyria in a subject include, without limitation, one or more of the following: (i) the loss of detectability of the mutated gene; (ii) increase in time to disease relapse; (iii) loss of photosensitivity for those with X-linked dominant protoporphyria (XLDPP), congenital erythropoietic porphyria (CEP), porphyria cutanea tarda (PCT), harderoporphyria (HP), variegate porphyria (VP), and/or erythropoietic protoporphyria (EPP); (iv) loss of peripheral neuropathy for those with aminolevulinate dehydratase deficiency porphyria (ALADP); and (v) loss of peripheral neuropathy for those with acute intermittent porphyria (AIP).

In that regard, this invention further provides a composition comprising (i) a selenium-based agent and (ii) a therapeutic agent that can exacerbate porphyria in a subject whose cells possess none, one, or more mutations in the heme biosynthesis pathway. The following are some examples of the present composition: (i) a selenium-based agent (e.g., sodium selenite pentahydrate) and a classic antiepileptic drug (e.g., phenobarbital, phenytoin, carbamazepine, or primidone); (ii) a selenium-based agent (e.g., sodium selenite pentahydrate) and a calcium channel blocker (e.g., nifedipine or israpidine); (iii) a selenium-based agent (e.g., sodium selenite pentahydrate) and an antibiotic (e.g., sulfadiazine, sulfamethoxazole, or rifampicin); (iv) a selenium-based agent (e.g., sodium selenite pentahydrate) and a fungicide (e.g., ketoconazole); and (v) a selenium-based agent (e.g., sodium selenite pentahydrate) and a reproductive steroid (e.g., progesterone, medroxyprogesterone, or testosterone).

This invention still further provides a method for treating a human subject having RARS comprising orally administering to the subject an effective amount of sodium selenite (e.g., sodium selenite pentahydrate).

Finally, this invention provides certain articles of manufacture. Specifically, this invention provides an article of manufacture comprising (i) a selenium-based agent, (ii) a pharmaceutically acceptable carrier, and (iii) a label indicating a use for an admixture of the agent and carrier in treating a subject afflicted with RARS.

This invention also provides an article of manufacture comprising (i) sodium selenite (e.g., sodium selenite pentahydrate), (ii) a pharmaceutically acceptable carrier, and (iii) a label indicating a use for an admixture of the sodium selenite and carrier in treating a human subject having RARS.

This invention further provides an article of manufacture comprising (i) a selenium-based agent, (ii) a pharmaceutically acceptable carrier, and (iii) a label indicating a use for an admixture of the agent and carrier in treating a subject having a cancer characterized by one or more genetic mutations associated with ring sideroblasts.

This invention still further provides an article of manufacture comprising (i) sodium selenite (e.g., sodium selenite pentahydrate), (ii) a pharmaceutically acceptable carrier, and (iii) a label indicating a use for an admixture of the sodium selenite and carrier in treating a subject having a cancer is selected from the group consisting of uveal melanoma, chronic lymphoid leukemia, melanoma, acute myeloid leukemia, breast cancer, pancreatic cancer, non-small cell lung cancer, and cervical cancer.

The present methods, compositions, and articles of manufacture are envisioned for treating tyrosinemia, mutatis mutandis, as they are for treating RARS in this invention.

This invention will be better understood by reference to the examples which follow, but those skilled in the art will readily appreciate that the specific examples detailed are only illustrative of the invention as described more fully in the claims which follow thereafter.

### EXAMPLES

### Example 1 - Treatment of Intermediate or High Risk MDS (Including RARS)

For an adult diagnosed with intermediate or high risk MDS (which includes RARS), treat the patient with (i) 250 mg/m² intravenous sodium selenite in conjunction with the standard seven-day dosing regimen of azacytidine for seven days every 28 days, and (ii) 2,000 µg daily oral sodium selenite for the duration of the treatment period.

### Example 2 - Treatment of CLL

For an adult diagnosed with CLL, treat the patient with (i) 250 mg/m² intravenous sodium selenite (preferably sodium selenite pentahydrate) in conjunction with the intravenous delivery of FCR components; and (ii) 2,000 µg daily oral sodium selenite (e.g., sodium selenite pentahydrate) for the duration of the treatment period.

FCR is administered according to the following regimen. Cycle 1 of FCR consists of the following: rituximab 375 mg/m² IV at 50 mg/h (increase by 50 mg/h every 30 minutes until a target rate of 400 mg/h is reached) on day 1, plus cyclophosphamide 250 mg/m² IV over 10-30 minutes on days 1-3, plus fludarabine 25 mg/m² IV over 20-30 min on days 1-3. Cycles 2-6 of FCR differ from cycle 1 only in that the rituximab dose is increased from 375 mg/m² to 500 mg/m². Thus, the regimen is as follows: rituximab 500 mg/m² IV at 50 mg/h (increase by 50 mg/h every 30 minutes until a target rate of 400 mg/h is reached) on day 1, plus cyclophosphamide 250 mg/m² IV over 10-30 minutes on days 1-3, plus fludarabine 25 mg/m² IV over 20-30 minutes on days 1-3. In conjunction with FCR administration, the selenium-based agent (e.g., sodium selenite pentahydrate) is administered, for example, (i) intravenously as a saline solution immediately following FCR administration, (ii) orally on all days of FCR administration, or (iii) intravenously daily at high concentration for three days at the beginning of, or later during, FCR administration.

### Example 3 - Treatment of Porphyria

For an adult diagnosed with a form of porphyria, treat the patient with a thrice daily oral dose of 1,500 µg sodium selenite pentahydrate daily for one year.

### Example 4 - Sodium Selenite Effect on Mutant Cancer Cells

### Synopsis

This experiment shows that when melanoma and leukemia cells, genetically manipulated by transfection with the splicing factor mutation K700E, are grown in the presence of sodium selenite, sodium selenite has a cytotoxic effect against the mutated cells. Briefly, in this experiment, cell lines representing melanoma (MEL1) and acute myeloid leukemia (KG1) were transfected either with a silent mutation (a, c) or an oncogenic splicing mutation K700E (b, d) and grown in 20 µM sodium selenite pentahydrate. Cells were cultured in duplicate and survival was measured by luminescence on a TecanⓇ plate reader with Cell Titer Glo 2.0® (Promega). The results are shown in FIG. 1.

### Methods

Plasmid and ssODN DNA Prep. Plasmid DNA containing the sequence of the genome-editing protein Cas9 and its genomic guide sequence targeting the SF3B1 K700E mutation was prepared by GenScript® (Piscataway, NJ) and diluted to a final concentration of 1 µg/µL in deionized nuclease-free water. Single-stranded oligo transcripts that act as repair templates during homology driven repair were purchased from Integrated DNA Technologies (IDT, Coralville, IA) and added to the plasmid solution to a final concentration of 25 µM (see below for transcript sequences).

Cell Culture. Melanoma cells (ATCC #SK-MEL-1) were cultured at 37° C. with 5.0% CO₂ in ATCC-formulated Eagle’s Minimum Essential Medium (ATCC 30-2003) containing fetal bovine serum (Life Technologies #26140079) to a final concentration of 10%. Cells were established at 1 × 10⁵ cells/mL and maintained by addition or replacement of fresh medium at 2 × 10⁵ cells/mL every two to three days. KG1-a cells (ATCC CCL-246.1 ™) were cultured at 37° C. with 5.0% CO₂ in ATCC-formulated Iscove’s Modified Dulbecco’s Medium (ATCC 30-2005), containing fetal bovine serum (Life Technologies #26140079), to a final concentration of 20%. Cells were established at 2 × 10⁵ viable cells/mL and maintained by addition or replacement of fresh medium at 2 × 10⁵ cells/mL every two to three days.

Transfection. After three passages, cells were collected when 70-90% confluent, washed in phosphate-buffered saline (PBS) without Ca2+ and Mg2+, and resuspended in Buffer R Resuspension Buffer (included with Neon™ Kits) to a final density of 1.0 × 10⁷ cells/mL. Using a Neon™ pipette (included with Neon™ Kits), 100 µL of cells were mixed with 10 µL of plasmid/template DNA and MEL lines were transfected with one pulse of 1,000 volts for 40 milliseconds and KG1 lines with one pulse of 1,700 volts for 20 milliseconds with a Neon Transfection System (Life Technologies MPK5000) before a 48-hour incubation at 37° C. with 5.0% CO₂ in sterile 6-well tissue culture plates (Stellar #TC1 0-006) containing 2 mL of fresh media.

Sorting. Cells were washed in PBS, resuspended in PBS with 2% FBS, and sorted for GFP fluorescence by a BD FACSArialll® flow cytometer (BD Biosciences, Franklin, NJ). Fluorescent cells were selected and transferred to a solid black 96-well tissue culture plates (Fisher #08-772-225) at a density of 200,000 cells/mL and cultured for 48 hours with or without 20 µM Na-selenite pentahydrate (Sigma #S5261).

Drug Screening. One sample volume of Cell Titer Glo 2.0® (PromegaⓇ #G9241 ) was added to each sample well and after a 2-minute incubation on an orbital shaker followed by a 10-minute incubation, chemiluminescence was read by a TecanⓇ Infinite F500 Multimode Microplate Reader (Tecan Trading AG, Zurich, CH).

DNA Templates. K700K ssODN Repair Template: CAATGGCCAAAGCACTG-ATAGTCCGAACCTTCTGCTGCTCATCCACAAGA. K700E ssODN Repair Template: CAATGGCCAAAGCACTGATAGTCCGAACTTCCTGCTGCTCA-TCCACAAGA. Guide RNA Target Sequence, px458 PAM1_pSpCas9 BB-2A-GFP (PX458) gRNA: AGCAATGGCCAAAGCACTGA. Cas9 Plasmid Sequence with gRNA, px458 PAM1_pSpCas9 BB-2A-GFP (PX458): see FIG. 2.

### References

Allen, N.E., et al., Plasma selenium concentration and prostate cancer risk: results from the European Prospective Investigation into Cancer and Nutrition (EPIC). Am. J. Clin. Nutr. 2008;88:1567-75.

Bjelakovic, G., et al., Systematic review: primary and secondary prevention of gastrointestinal cancers with antioxidant supplements. Aliment Pharmacol. Ther. 2008;28:689-703.

Brinkman, M., et al., Are men with low selenium levels at increased risk of prostate cancer? Eur. J. Cancer 2006;42:2463-71,

Chen, Y.C., et al., Is selenium a potential treatment for cancer metastasis?, Nutrients 2013, 5:1149-1168.

Combs, G.F., Jr. and Gray, W.P., Chemopreventive agents: Selenium. Pharmacol. Ther. 1998; 79:179-92.

Dennert, G., et al., Selenium for preventing cancer. Cochrane Database Syst. Rev 2011 :CD005195.

Davis, C.D., Selenium supplementation and cancer prevention. Curr. Nutr. Rep. 2012;1:16-23.

Duffield-Lillico, A.J., et al., Selenium supplementation, baseline plasma selenium status and incidence of prostate cancer: an analysis of the complete treatment period of the Nutritional Prevention of Cancer Trial. BJU Int. 2003;91 :608-12.

Gonçalves, A.C., Selenium compounds induced ROS-dependent apoptosis in myelodysplasia cells, Biol. Trace Elem. Res., 2013 Sep;154(3):440-7.

Institute of Medicine, Food and Nutrition Board (IMFN Board). Dietary Reference Intakes: Vitamin C, Vitamin E, Selenium, and Carotenoids. National Academy Press, Washington, DC, 2000.

Klein, E.A., et al., Vitamin E and the risk of prostate cancer: the Selenium and Vitamin E Cancer Prevention Trial (SELECT). JAMA 2011;306:1549-1556.

Lippman, S.M., et al., The effect of selenium and vitamin E on risk of prostate cancer and other cancers: the Selenium and Vitamin E Cancer Prevention Trial (SELECT). JAMA 2009;301:39-51.

MacFarquhar, J.K., et al., Acute selenium toxicity associated with a dietary supplement. Arch. Intern. Med. 2010;170:256-61.

Malcovati, L., et al., SF3B1 mutation identifies a distinct subset of myelodysplastic syndrome with ring sideroblasts, Blood, 2015 Jul. 9;126(2):233-41.

Rayman, M.P., Selenium and human health. Lancet 2012;379:1256-68.

Sunde, R.A., Selenium. In: Bowman, B., and Russell, R., eds., Present Knowledge in Nutrition. 9th ed. Washington, DC: International Life Sciences Institute; 2006:480-97.

Sunde, R.A., Selenium. In: Coates, P.M., et al., eds. Encyclopedia of Dietary Supplements. 2nd ed. London and New York: Informa Healthcare; 2010:711-8.

Sunde, R.A., Selenium. In: Ross, A.C., et al., eds. Modern Nutrition in Health and Disease. 11th ed. Philadelphia, PA: Lippincott Williams & Wilkins; 2012:225-37.

Terry, E.N., and Diamond, A.M., Selenium. In: Erdman, J.W., et al., eds., Present Knowledge in Nutrition. 10th ed. Washington, DC: Wiley-Blackwell; 2012:568-87.

U.S. Food and Drug Administration. Qualified Health Claims: Letters of Enforcement Discretion (2017).

Vinceti, M., et al., Selenium for preventing cancer. Cochrane Database of Systematic Reviews 2018, Issue 1. Art. No.: CD005195.

1. A method for treating a subject having refractory anemia with ring sideroblasts (RARS) comprising administering to the subject an effective amount of a selenium-based agent.

2. A method for treating a subject having a cancer characterized by one or more genetic mutations associated with ring sideroblasts, comprising administering to the subject an effective amount of a selenium-based agent.

3. The method of claim 2, wherein the cancer is characterized by one or more mutations in the SF3B1 gene.

4. The method of claim 2, wherein the cancer is characterized by one or more SF3B1 mutations selected from the group consisting of a K700 mutation, a K666 mutation, an H662 mutation, an E622 mutation, and an R625 mutation.

5. The method of claim 2, wherein the cancer is selected from the group consisting of uveal melanoma, chronic lymphoid leukemia, melanoma, acute myeloid leukemia, breast cancer, pancreatic cancer, non-small cell lung cancer, and cervical cancer.

6. A method for treating a subject having porphyria comprising administering to the subject an effective amount of a selenium-based agent.

7. The method of claim 6, wherein the porphyria is selected from the group consisting of porphyria cutanea tarda, acute intermittent porphyria, and erythropoietic protoporphyria.

8. A method for inhibiting the exacerbation of porphyria in a subject to whom a therapeutic agent is administered, wherein the subject’s cells possess one or more mutations in the heme biosynthesis pathway that render the subject susceptible to porphyria exacerbation by the therapeutic agent, which method comprises administering to the subject an effective amount of a selenium-based agent before, during, and/or after administering the therapeutic agent.

9. The method of claim 1, wherein the selenium-based agent is sodium selenite.

10. The method of claim 1, wherein the subject is a human.

11. A method for treating a human subject having refractory anemia with ring sideroblasts (RARS) comprising orally administering to the subject an effective amount of sodium selenite.

12-15. (canceled)
